(19) United States
(12) Patent Application Publication
(10) Pub. No.: US 2002/0006435 A1
(43) Pub. Date: Jan. 17, 2002

(54) Transdermal anesthetic and vasodilator composition and methods for topical administration

(76) Inventors: Paul J. Samuels, Cincinnati, OH (US); Dawn Sweeney, Ft. Thomas, KY (US)

Correspondence Address: Frost Brown Todd LLC, 2200 PNC Center, 201 East Fifth Street, Cincinnate, OH, 45202-4182, US

(21) Appl. No.: 09/770,344

(22) Filed: Jan. 26, 2001

Related U.S. Application Data
(60) Provisional application No. 60/178,364, filed on Jan. 27, 2000.

(51) Int. Cl. A61K 9/70; A61K 31/46; A61K 31/137

(52) U.S. Cl. 424/449; 514/654; 514/304

(57) ABSTRACT

A composition for topical application comprising a therapeutically effective amount of a topical anesthetic, a safe and effective amount of a pharmaceutically acceptable topical vasodilator and a pharmaceutically acceptable carrier and a method of administering the composition to a mammal are disclosed.

### CROSS-REFERENCE TO RELATED APPLICATION

This application is based on and claims priority from U.S. Provisional Patent Application Serial No. 60/178,364, filed Jan. 27, 2000, which application is hereby incorporated by reference in its entirety.

### FIELD OF THE INVENTION

The present invention relates to compositions and methods for the topical administration of pharmaceutically active anesthetics to a mammal in need thereof. More particularly, the present invention relates to local anesthetic agents for topical administration combined with pharmaceutical agents having vasodilator activity. The present invention also relates to method of inserting an intravenous line into a patient comprising the steps of applying a safe and effective amount of the anesthetic composition to the skin of the patient at the site of insertion to prevent or ameliorate pain as well as to facilitate intravenous line insertion.

### DESCRIPTION OF BACKGROUND ART

Anesthetic agents are pharmacologically active agents that block nerve conduction when applied in therapeutically effective amounts. Anesthetic agents have been used extensively to obtain topical anesthesia. Topical administration or application means the direct contact of the anesthetic with tissue such as skin or membrane. Previous method of applying topical anesthetic agents to the skin have used non-finite or semi-liquid carriers such as gels or ointments or finite carriers such as non-spreading substances which retain their form such as patches, dressings and bandages.

To be effective, a topical local anesthetic should contain sufficient concentration of the active agent to produce an anesthetic effect. Generally it is well know to use topical anesthetics and transdermal anesthetics are known to be useful for numbing an area prior to venapuncture, such as blood drawing, or intravenous line insertion.

Lidocaine is highly effective and is the most commonly used local anesthetic especially in the form of aqueous solutions of lidocaine hydrochloride, which are administered intravenously. Lidocaine is also formulated as a jelly ointment or spray for use as an anesthetic. Unfortunately, these formulations are only effectively absorbed through mucosal surfaces and not the skin. A more recently developed transdermal anesthetic that utilizes lidocaine is EMLA(® cream (eutectic mixture of local anesthetics) which patients have found preferable to lidocaine or ethylchloride spray. EMLA® cream is commercially available from Astra USA, Inc., Westborro, Mass. EMLA® cream is an oil and water emulsion in which the oil phase is a eutectic mixture of lidocaine and prilocaine in a ratio of 1:1 by weight (2.5% and 2.5%, respectively) and comprising 92% purified water. A eutectic mixture is a mixture is a mixture that has a melting point lower than that of its ingredients. Therefore, the two anesthetics after being heated and mixed exist as a liquid oil at room temperature rather than as crystals. EMLA® cream is described in U.S. Pat. Nos. 4,529,601 and 4,562,060, which teach the mixing of specific portions of certain local anesthetic agents in the form of their base in order to form a homogenous oil having a melting point below 40° C., which are hereby incorporated by reference in their entirety. Like lidocaine, prilocaine is an amide type local anesthetic agent.

Commonly, prolongation of anesthesia with topical anesthetics has been achieved by the addition of vasoconstrictors such as catecholamines like epinephrine. Vasoconstrictors cause constriction of blood vessels and have the potential of adverse side effects.

U.S. Pat. No. 5,993,836, to Castillo, describes a topical transdermal anesthetic comprising a eutectic mixture of lidocaine and prilocaine incorporated within a lipophilic base. These anesthetic formulations have significantly more rapid onset than comparable transdermal anesthetics such as EMLA® cream.

U.S. Pat. No. 5,942,543, to Ernst, relates to topical anesthetic preparations comprising lidocaine, adrenaline and tetracaine along with methods of use. The adrenaline (or epinephrine) added is a catecholamine, which acts as a vasoconstrictor.

It has now been found that the addition of a vasodilator can be added to a topical anesthetic to aid in venapuncture or intravenous line insertion when applied in a safe and effective amount to the skin of a patient at the site of insertion. A vasodilator is a substance that causes dilation of blood vessels when administered transdermally, intravenously or orally.

### SUMMARY OF THE INVENTION

It has now been surprisingly discovered that by incorporating a vasodilator into a composition comprising transdermal anesthetics in a pharmaceutically acceptable carrier, a transdermal formulation is produced which has both anesthetic and vasodilator properties.

The present invention relates to a composition for topical application comprising:

a. a therapeutically safe and effective amount of at least one topical anesthetic;

b. a therapeutically safe and effective amount of at least one topical vasodilator; and

c. a pharmaceutically acceptable carrier.

In another embodiment, the composition of the invention is comprised of at least two topical anesthetic agents. The present invention further relates to a composition comprising from about 0.5 to about 20% of a topical anesthetic agent from about 0.01 to about 1% of a vasodilator agent with the balance of the composition being a pharmaceutically acceptable carrier.

The invention further relates to the a method of administering topical anesthetic agents along with a vasodilator comprising the steps of:

a. providing a composition comprising a therapeutically safe and effective amount of at least one topical anesthetic; a therapeutically safe and effective amount of at least one vasodilator; and a pharmaceutically acceptable carrier for the anesthetic and vasodilator; and

b. contacting an area of skin with the composition thereby administering the topical anesthetic and vasodilator preferably at the site of insertion of an intravenous device.

### DETAILED DESCRIPTION OF THE INVENTION

The present invention provides compositions that, when applied to an area of the skin, deliver a combination of pharmaceutical agents to produce a local effect over a period of time. In accordance with one embodiment of the present invention, a topical anesthetic substantially in solution with a topical vasodilator and pharmaceutically acceptable carrier is provided for application to the skin of a mammal.

The anesthetic agents of this invention are those known or of a type known in the art. The topical anesthetic agent suitable for the use in the practice of this invention include amides and esters of benzoic acid derivatives administered either as the free base or the acid addition salt. To be effective, a topical local anesthetic should contain sufficient concentration of the active agent to produce an anesthetic effect. It should penetrate intact skin sufficiently to deliver a therapeutic dose and it should exhibit rapid unset of anesthetic action and have a prolonged anesthetic effect.

The local anesthetic bases encompassed by this invention are weak organic bases that are lipophilic in nature and thus poorly soluble in water. However, these bases will react with organic or inorganic acids to form acidic water-soluble acid addition salts. Thus the term “base” as used herein means the unionized form of the anesthetic that can furnish an electron pair to form a covalent bond. The term “acid” as used herein is a substance that can take up an electron pair to form a covalent bond. The term “salt” as used herein means the form produced by a base upon its reaction with an organic or inorganic acid.

As described herein, and in the accompanying documents, the present invention encompasses topical anesthetic compositions comprising a safe and effective amount of a topical anesthetic, such as a eutectic mixture of lidocaine and prilocaine; a safe and effective amount of a topical vasodilator, such as nitroglycerine, and a pharmaceutically acceptable carrier. The present invention relates to a composition for topical application comprising:

a. a therapeutically safe and effective amount of at least one topical anesthetic;

b. a therapeutically safe and effective amount of at least one topical vasodilator; and

c. a pharmaceutically acceptable carrier.

The present invention further relates to a composition comprising from about 0.5% to about 30% of a topical anesthetic agent and from about 0.01% to about 5% of a vasodilator agent with the balance of the composition being a pharmaceutically acceptable carrier. Preferably, the composition comprises from about 0.5% to about 20% of a topical anesthetic agent and from about 0.01% to about 2% of a vasodilator agent. Most preferably, the composition comprising from about 0.5% to about 10% of a topical anesthetic agent and from about 0.01% to about 1% of a vasodilator agent. In another embodiment, the composition of the invention is comprised of at least two topical anesthetic agents.

The invention further relates to the a method of administering topical anesthetic agents along with a vasodilator comprising the steps of:

a. providing a composition comprising a therapeutically safe and effective amount of at least one topical anesthetic; a therapeutically safe and effective amount of at least one vasodilator; and a pharmaceutically acceptable carrier for the anesthetic and vasodilator; and

b. contacting an area of skin with the composition thereby administering the topical anesthetic and vasodilator preferably at the site of insertion of an intravenous device.

The present invention further encompasses waiting an appropriate time for the composition to provide its anesthetic effect (generally 30-60 minutes); and then inserting the I.V. line at the site of application.

The term “safe and effective amount” as used herein, means an amount of an active ingredient high enough to deliver the desired skin benefit, but low enough to avoid serious side effects, at a reasonable benefit to risk ratio within the scope of sound medical judgement. What is a safe and effective amount of the active ingredient will vary with the specific active, the ability of the active to penetrate through the skin, the age, health condition, and skin condition of the user, and other like factors.

The term “pharmaceutically-acceptable” as used herein, means any of the commonly-used materials that are suitable for use in contact with the tissues of humans without undue toxicity, irritation, incompatibility, instability, irritation, allergic response, and the like.

Anesthetic

This invention is applicable to any topical anesthetic, especially those with vasoconstrictive properties. Examples of topical anesthetic drugs include benzocaine, bupivacaine, butamben, butambenpicrate, chloroprocaine hydrochloride, chlorprocaine, cocaine, cocaine hydrochloride, dibucaine, dibucaine hydrochloride, dimethisoquin hydrochloride, diperodon, diphenhydramine, dyclonine, dyclonine hydrochloride, etidocaine, hexylcaine, ketamine, lidocaine, lidocaine hydrochloride, mepivacaine, methapyriline, oxyprocaine hydrochloride, p-buthylaminobenzoic acid 2-(die-ethylamino) ethyl ester hydrochloride, phenol, piperocaine hydrochloride, pramoxine, pramoxine hydrochloride., procaine, procaine hydrochloride, tetracaine, tetracaine hydrochloride, tripelennamine, and pharmaceutically acceptable acids, bases, and salts thereof. These anesthetics may be used individually or in mixtures.

Preferably, the topical anesthetic is at least one pharmaceutically active anesthetic selected from the group consisting of benzocaine, lidocaine, bupivacaine, dibucaine, mepivacaine, etidocaine, butanilicaine, prilocaine, tetracaine and trimecaine and their salts thereof. More preferably, the topical anesthetic is at least one pharmaceutically active anesthetic selected from the group consisting of lidocaine, dibucaine, tetracaine, and prilocaine and their salts thereof. Most preferably, the topical anesthetic is at least one pharmaceutically active anesthetic selected from the group consisting of lidocaine and prilocaine and their salts thereof. Preferably, the anesthetic is a eutectic mixture of topical anesthetics.

The acid-addition salts of the present invention are any non-toxic, pharmaceutically acceptable organic or inorganic salts. Typical inorganic salts are the hydrogen halides, especially the hydrochlorides, carbonates, borates, phosphates, sulfates, hydrogen sulfates, hydrobromides, nitrates, sulfides, and arsenates. Typical organic salts are salts of mono- and polycarboxylic acids such as the citrate, tartrate, malate, cinnamate, oxalate, formate, succinate and phthalates. A preferred salt is the hydrochloride.

In another embodiment, the local topical, transdermal anesthetic comprises a eutectic mixture of at least one first pharmaceutically active anesthetic and at least one second pharmaceutically active anesthetic in a ratio of about 20:1 to about 1:20, wherein at least one first pharmaceutically active anesthetic is selected from the group consisting of benzocaine, lidocaine, bupivacaine, dibucaine, mepivacaine, etidocaine, tetracaine, butanilicaine and trimecaine and at least one second pharmaceutically active anesthetic is selected from the group consisting of prilocaine, tetracaine, butanilicaine and trimecaine, and wherein the at least one first pharmaceutically active anesthetic is different than the at least one second pharmaceutically active anesthetic.

Preferably, the anesthetic comprises a eutectic mixture of at least one first pharmaceutically active amide type anesthetic and at least one second pharmaceutically active amide type anesthetic in a ratio of about 15:1 to about 1:15. More preferably, the anesthetic comprises a eutectic mixture of at least one first pharmaceutically active anesthetic and at least one second pharmaceutically active anesthetic in a ratio of about 10:1 to about 1:10. Most preferably, the anesthetic comprises a eutectic mixture of at least one first pharmaceutically active anesthetic and at least one second pharmaceutically active anesthetic in a ratio of about 4:1 to about 1:4.

In another embodiment, the anesthetic agent comprises a eutectic mixture of lidocaine and prilocaine. Preferably, the anesthetic agent comprises a eutectic mixture consisting essentially of prilocaine in the form of its base in admixture with lidocaine, in the form of its base in a weight ratio of from about 1:4 to about 4:1. Generally, such eutectic mixtures will be in a lipophilic base comprising from 30% to 90% by weight of the formulation. In another embodiment, the topical anesthetic is a eutectic mixture of lidocaine, prilocaine and dibucaine.

The most commonly used such topical anesthetic is EMLA Cream, commercially available from Astra USA, Inc., Westborough, Mass. EMLA Cream is an emulsion in which the oil phase is a eutectic mixture of lidocaine and prilocaine in a ratio of 1:1 by weight. A eutectic mixture has a melting point below room temperature and, therefore, both anesthetics exist as a liquid oil, rather than as crystals.

Lidocaine is chemically designated as 2-(di-ethylamino)-N-(2,6-dimethylphenyl) acetamide, and has a molecular weight of 234.3 (C₁₄H₂₂N₂O). Prilocaine is chemically designated as N-(2-methylphenyl)-2-(propylamino) propanamide, and has a molecular weight of 220.3 (Cl₃H₂₀N₂O). Each gram of EMLA Cream contains lidocaine 25 mg, prilocaine 25 mg, polyoxyethylene fatty acid esters (as emulsifiers), carboxypolymethylene (as a thickening agent), sodium hydroxide to adjust the composition to a pH of about 9, and purified water (about 92%).

Vasodilator

Any vasodilator effective upon topical administration may be used in the present invention. “Vasodilator” includes any substance that causes dilation of blood vessels. Examples of suitable vasodilators include nitroglycerine, glyceryl trinitrate, adenine, arginine, phentolamine, nicotinates, various prostaglandins (eicosanoids), various calcium antagonists, papaverine, nimodipine, hydralazine, nitric oxide, epoprostenol, tolazoline, arninone, milrinone, nitroglycerine, isosorbide dinitrate, isosorbide mononitrate, erythritol tetranitrate, pentaerythritol tetranitrate, dipyridamole, dilazep, trapidil, trimetazidine, and their substituted derivatives.

In addition to trinitrin (nitroglycerine), the following are examples of organic nitrates useful as vasodilators: tetranitroerythritol, hexanitroinositol, tetranitropentaerythritol, propatyl nitrate, isosorbide 5-mononitrate (IS-5-MN), isosorbide dinitrate, isosorbide 2-mononitrate (IS-2-MN), isomannide 2-nitrate and trinitrotriethanolamine, and their substituted derivatives, in particular the aminopropanol derivatives of 1,4:3,6-dianhydrohexitol nitrates.

A preferred vasodilator for use in the present invention is nitroglycerine, for example, commercially available under the tradename Nitroglycerine Ointment, 2%, from Fougera. Nitroglycerine is chemically designated as 1,2,3-propanetriol trinitrate, having a molecular weight of 227.1. Topical formulations typically utilize lanolin and/or petrolatum as the vehicle.

Carrier

The compositions of the present invention comprise a safe and effective amount of a dermatologically acceptable carrier within which the essential particulate material and optional other materials are incorporated to enable the particulate material and optional components to be delivered to the skin at an appropriate concentration. The carrier can thus act as a diluent, dispersant, solvent, or the like for the particulate material which ensures that it can be applied to and distributed evenly over the selected target at an appropriate concentration.

The carrier may contain one or more dermatologically acceptable solid, semi-solid or liquid fillers, diluents, solvents, extenders and the like. The carrier may be solid, semi-solid or liquid. Preferred carriers are substantially liquid. The carrier can itself be inert or it can possess dermatological benefits of its own. Concentrations of the carrier can vary with the carrier selected and the intended concentrations of the essential and optional components.

Suitable carriers include conventional or otherwise known carriers that are dermatologically acceptable. The carrier should also be physically and chemically compatible with the essential components described herein, and should not unduly impair stability, efficacy or other use benefits associated with the compositions of the present invention. Preferred components of the compositions of this invention should be capable of being comingled in a manner such that there is no interaction that would substantially reduce the efficacy of the composition under ordinary use situations.

The type of carrier utilized in the present invention depends on the type of product form desired for the composition. The topical compositions useful in the subject invention may be made into a wide variety of product forms such as are known in the art. These include, but are not limited to, lotions, creams, gels, sticks, sprays, ointments, pastes, and the like. These product forms may comprise several types of carriers including, but not limited to, solutions, aerosols, emulsions, gels, solids, and liposomes.

Preferred carriers contain a dermatologically acceptable, hydrophilic diluent. As used herein, “diluent” includes materials in which the particulate material can be dispersed, dissolved, or otherwise incorporated. Nonlimiting examples of hydrophilic diluents are water, organic hydrophilic diluents such as lower monovalent alcohols (e.g., C1-C4) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

Preferred carriers comprise an emulsion comprising a hydrophilic phase comprising a hydrophilic component, e.g., water or other hydrophilic diluent, and a hydrophobic phase comprising a hydrophobic component, e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the composition ingredients. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may be or comprise (e.g., in a triple or other multi-phase emulsion) an oil-in-water emulsion or a water-in-oil emulsion such as a water-in-silicone emulsion.

The topical compositions of the subject invention, including but not limited to lotions and creams, may comprise a dermatologically acceptable emollient. Emollients tend to lubricate the skin, increase the smoothness and suppleness of the skin, prevent or relieve dryness of the skin, and/or protect the skin. Emollients are typically water-immiscible, oily or waxy materials. A wide variety of suitable emollients are known and may be used herein.

Lotions and creams according to the present invention generally comprise a solution carrier system and one or more emollients. Lotions typically comprise from about 1% to about 20%, preferably from about 5% to about 10%, of emollient; from about 50% to about 90%, preferably from about 60% to about 80%, water. A cream typically comprises from about 5% to about 50%, preferably from about 10% to about 20%, of emollient; and from about 45% to about 85%, preferably from about 50% to about 75%, water.

Ointments of the present invention may comprise a simple carrier base of animal or vegetable oils or semi-solid hydrocarbons (oleaginous); absorption ointment bases which absorb water to form emulsions; or water soluble carriers, e.g., a water soluble solution carrier. Ointments may further comprise a thickening agent, such as described in Sagarin, Cosmetics, Science and Technology, 2nd Edition, Vol. 1, pp. 72-73 (1972), incorporated herein by reference, and/or an emollient. For example, an ointment may comprise from about 2% to about 10% of an emollient, and from about 0.1% to about 2% of a thickening agent.

Any compatible topical carrier may be used in the compositions of the present invention. The compositions can, for example, be formulated as creams, lotions, solutions, gels or sprays. The carrier may include, for example, emollients, emulsifiers, thickening agents, buffers, solvents, mixed solvents, preservatives, coloring agents, fragrances, anti-oxidants, preservatives antimicrobial and antifungal actives, anti-inflammatory actives, lower alcohols, polyols, esters of fatty acids, oils, and waxes, silicones, antifoam agents, hydrating agents, stabilizers, surfactants, fillers, sequestrants, anionic, cationic, nonionic and amphoteric polymers, propellants, alkalifying and acidifying agents and mixtures thereof.

Optional Ingredients

The compositions of the present invention can comprise a wide variety of optional components. Typical of such optional components are:

Thickeners.

The compositions of the present invention can also comprise a thickening agent, preferably from about 0.1% to about 10%, more preferably from about 0.1% to about 5%, and most preferably from about 0.2% to about 3%, of a thickening agent. Thickeners that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL” (B.F. Goodrich, Cleveland, Ohio), “HYPAN” (Kingston Technologies, Dayton, N.J.), “NATROSOL” (Aqualon, Wilmington, Del.), “KLUCEL” (Aqualon, Wilmington, Del.), or “STABILEZE” (ISP Technologies, Wayne, N.J.).

Other preferred gelling polymers include carboxypolymethylene, hydroxyethylcellulose, cellulose gum, MVE/MA decadiene crosspolymer, PVM/MA copolymer, or a combination thereof.

Preservatives.

Preservatives may also be used in this invention and preferably comprise from about 0.001% to about 0.5% by weight of the total composition. The use of preservatives assures that if the product is microbially contaminated, the formulation will prevent or diminish microorganism growth. Some preservatives useful in this invention include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof. Moreover, BHA may be used as an antioxidant, as well as to protect ethoxydiglycol and/or dapsone from discoloration due to oxidation. An alternate antioxidant is BHT.

Vehicles or excipients.

Vehicles or excipients that may be used in the composition of the invention include any non toxic non aqueous compound such as an oil such as paraffin which are suitable for topical application and which are liquid at room temperature. Paraffins may comprise purified clear, oily, tasteless and odorless mixtures of saturated aliphatic or cycloaliphatic hydrocarbons e.g. low viscosity or viscous paraffin such as sold under the Registered Trade Mark VASELINE. Other preferred excipients for percutaneous delivery include those which are suitable for the preparation of creams, liniments, ointments, aerosol sprays, gels, pastes or foams suitably containing from about 0.5% to about 20% of active agents in aqueous phase of viable epidermis.

Antimicrobial and antifungal actives.

Antimicrobial and antifungal actives that may be used in the composition of the invention include: beta.- lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate and clotrimazole.

Surfactants.

The emulsion may contain an emulsifier and/or surfactant, generally to help disperse and suspend the discontinuous phase within the continuous phase. A wide variety of such agents can be employed. In one embodiment, the compositions of the present invention comprise from about 0.05% to about 10%, preferably from about 1% to about 6%, and more preferably from about 1% to about 3% of at least one surfactant which can disperse the materials in the water phase. The surfactant, at a minimum, must be hydrophilic enough to disperse in water. The surfactants useful herein can include any of a wide variety of cationic, anionic, zwitterionic, and amphoteric surfactants disclosed in prior patents and other references. The exact surfactant chosen will depend upon the pH of the composition and the other components present.

In one embodiment, the composition comprises a hydrophilic emulsifier or surfactant. The compositions of the present invention preferably comprise from about 0.05% to about 5%, more preferably from about 0.05% to about 2% of at least one hydrophilic surfactant. Without intending to be limited by theory, it is believed that the hydrophilic surfactant assists in dispersing hydrophobic materials, e.g., hydrophobic structuring agents, in the hydrophilic phase. The surfactant, at a minimum, must be hydrophilic enough to disperse in the hydrophilic phase. The exact surfactant chosen will depend upon the pH of the composition and the other components present.

Preferred hydrophilic surfactants are selected from nonionic surfactants. Among the nonionic surfactants that are useful herein are those that can be broadly defined as condensation products of long chain alcohols, e.g. C8-30 alcohols, with sugar or starch polymers, i.e., glycosides. These compounds can be represented by the formula (S)ₙ—O—R wherein S is a sugar moiety such as glucose, fiuctose, mannose, and galactose; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C8-20 alkyl group, and n is an integer of from about 1 to about 9. Commercially available examples of these surfactants include decyl polyglucoside and lauryl polyglucoside.

Other useful nonionic surfactants include the condensation products of alkylene oxides with fatty acids (i.e. alkylene oxide esters of fatty acids); the condensation products of alkylene oxides with 2 moles of fatty acids (i.e. alkylene oxide diesters of fatty acids); the condensation products of alkylene oxides with fatty alcohols (i.e. alkylene oxide ethers of fatty alcohols); and the condensation products of alkylene oxides with both fatty acids and fatty alcohols. Nonlimiting examples of these alkylene oxide derived nonionic surfactants include ceteth-6, ceteth-10, ceteth-12, ceteareth-6, ceteareth-10, ceteareth-12, steareth-6, steareth-10, steareth-12, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof.

Other nonionic surfactants suitable for use herein include sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, and mixtures thereof. Nonlimiting examples of these non-silicon-containing emulsifiers include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof.

Another emulsifier useful herein are fatty acid ester blends based on a mixture of sorbitan or sorbitol fatty acid ester and sucrose fatty acid ester, the fatty acid in each instance being preferably C8-C24, more preferably C10-C20. The preferred fatty acid ester emulsifier is a blend of sorbitan or sorbitol C16-C20 fatty acid ester with sucrose C10-C16 fatty acid ester, especially sorbitan stearate and sucrose cocoate.

The hydrophilic surfactants useful herein can alternatively or additionally include any of a wide variety of cationic, anionic, zwitterionic, and amphoteric surfactants such as are known in the art. The cationic surfactants useful herein include cationic ammonium salts such as quaternary ammonium salts, and amino-amides.

A wide variety of anionic surfactants are also useful herein. Nonlimiting examples of anionic surfactants include the alkoyl isethionates (e.g., C12-C30), alkyl and alkyl ether sulfates and salts thereof, alkyl and alkyl ether phosphates and salts thereof, alkyl methyl taurates (e.g., C12-C30), and soaps (e.g., alkali metal salts, e.g., sodium or potassium salts) of fatty acids.

Amphoteric and zwitterionic surfactants are also useful herein. Examples of amphoteric and zwitterionic surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 22 carbon atoms (preferably C8-C18) and one contains an anionic water solubilizing group, e.g., carboxy, sulfonate, sulfate, phosphate, or phosphonate. Examples are alkyl imino acetates, and iminodialkanoates and aminoalkanoates, imidazolinium and ammonium derivatives. Other suitable amphoteric and zwitterionic surfactants are those selected from the group consisting of betaines, sultaines, hydroxysultaines, alkyl sarcosinates (e.g., C12-C30), and alkanoyl sarcosinates.

Structuring Agent.

The compositions hereof, and especially the emulsions hereof, may contain a structuring agent. Structuring agents are particularly preferred in the oil-in-water emulsions of the present invention. Without being limited by theory, it is believed that the structuring agent assists in providing Theological characteristics to the composition which contribute to the stability of the composition. For example, the structuring agent tends to assist in the formation of the liquid crystalline gel network structures. The structuring agent may also function as an emulsifier or surfactant. Preferred compositions of this invention comprise from about 1% to about 20%, more preferably from about 1% to about 10%, most preferably from about 2% to about 9%, of one or more structuring agents.

Suitable structuring agents of the present invention are selected from the group consisting of palmitic acid, stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof. More preferred structuring agents of the present invention are selected from the group consisting of stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof. Even more preferred structuring agents are selected from the group consisting of stearic acid, palmitic acid, stearyl alcohol, cetyl alcohol, behenyl alcohol, steareth-2, and mixtures thereof.

Water.

The compositions of the present invention comprise from about 5% to about 98%, more preferably from about 10% to about 85%, and most preferably from about 10% to about 65%.

Alkoxylated Alcohols

The compositions of the present invention comprise from about 0.1% to about 25%, preferably from about 0.1% to about 15%, and more preferably from about 6% to about 10% of an alkoxylated alcohol and/or alkoxylated polyol.

Polypropylene Glycols

Polypropylene glycols and propylene glycol are useful herein, at a level of from about 1% to about 5% by weight of the composition, preferably from about 2% to about 3.5% by weight of the composition, to enhance the penetration of the acidic active ingredient of the present invention. Polypropylene glycols are polymers that are typically formed from the polymerization of propylene oxide, propylene glycol, propylchlorohydrin, propylbromohydrin, and other related materials. The polypropylene glycols are commonly designated as PPG's followed by a number indicating the average number of repeating units in the structure. For example, PPG-30 would correspond to the above structure wherein n has an average value of about 30. Based on this nomenclature, the polypropylene glycols useful herein encompass those designated as PPG-10 through PPG-50, more preferably those designated as PPG-15 through PPG40, and most preferably those designated as PPG-20 through PPG-34.

Humectants

Another optional component of the compositions of the present invention is a humectant. When used herein, the humectant can comprise from about 0.1% to about 20%, more preferably from about 0.5% to about 10%, and most preferably from about 1% to about 5% by weight of the composition. Even though these materials are defined herein as humectants, they can also possess moisturizing, skin conditioning, and other related properties.

Examples of humectants useful herein include materials such as urea; guanidine; saturated or unsaturated alkyl alpha hydroxy acids such as glycolic acid and glycolate salts (e.g. ammonium and quaternary allyl ammonium) and lactic acid and lactate salts (e.g. ammonium and quaternary alkyl ammonium); aloe vera in any of its variety of forms (e.g. aloe vera gel); polyhydroxy alcohols such as sorbitol, glycerol, low molecular weight polypropylene glycols (e.g., dipropylene glycol and tripropylene glycol), hexanetriol, propylene glycol, butylene glycol, hexylene glycol, and the like; polyethylene glycol; sugars and starches; sugar and starch derivatives (e.g., alkoxylated glucose); hyaluronic acid; chitin, starch-grafted sodium polyacrylates; lactamide monoethanolamine; acetamide monoethanolamine; propoxylated glycerol; and mixtures thereof.

Emollients

The compositions of the present invention can also include an emollient. Examples of suitable emollients include, but are not limited to, volatile and non-volatile silicone oils (e.g., dimethicone, cyclomethicone, dimethiconol, and the like), highly branched hydrocarbons, and mixtures thereof. The emollients can typically comprise in total from about 0.1% to about 50% by weight of the composition.

Anti-inflammatory agents.

Analgesic anti-inflammatory agents useful in the present invention include acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexemac piroxicam, phenylbutazone, oxyphenbutazone, clofezone, pentazocine, mepirizole, and the like;

Acetonide anti-inflammatory agents useful in the present invention include hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, and the like;

Vitamins.

Vitamins useful in the present invention include vitamins A, B, C, D, E and K and derivatives thereof, calciferols, mecobalamin, and the like for dermatological use. The composition may include vitamin E for the purpose of enhancing the penetration of therapeutically effective ingredients.

Neutralizing agents.

Neutralizing agents useful in the present invention include aqueous soluble basic materials. Illustrative nonlimiting examples include basic alkali metal salts and alkaline earth metal salts such as hydroxides and carbonates and basic amine compounds such as triethanolamine, isopropylamine and the like.

Additional Ingredients

A variety of additional ingredients can be incorporated into the compositions of the present invention. These additional ingredients, at a minimum, must be acid stable. Non-limiting examples of these additional ingredients include other thickening agents; saturated and/or unsaturated alkyl alpha hydroxy acids; resins; gums (e.g. guar gum, xanthan gum and the like); waxes (both naturally occurring and synthetic); polymers for aiding the film-forming properties and substantivity of the composition (such as a copolymer of eicosene and vinyl pyrrolidone; abrasive scrub particles for cleansing and exfoliating the skin; skin penetration aids; chelators and sequestrants; and aesthetic components such as fragrances, pigments, colorings, essential oils, skin sensates, astringents, skin soothing agents, skin healing agents and the like, nonlimiting examples of these aesthetic components include panthenol and derivatives (e.g. ethyl panthenol), aloe vera, pantothenic acid and its derivatives, clove oil, menthol, camphor, eucalyptus oil, eugenol, menthyl lactate, witch hazel distillate, allantoin, bisabalol, dipotassium glycyrrhizinate and the like.

The above listed compounds may be incorporated singly or in combination.

These compositions can span a broad range of consistencies from thin lotions to heavy creams. These compositions typically have viscosities ranging from about 100 cps to about 500,000 cps, preferably from about 3,000 cps to about 200,000 cps as measured at a temperature of 25° C. The compositions can span a wide range of pH values. Even though buffers can be utilized to help maintain the pH of the emulsion compositions, these are not required components, but are merely optional ingredients.

Methods of Use

The present invention also relates to method of treatment wherein a safe and effective amount of the active ingredients are deposited on the skin in order to modify the condition of the skin or dermis and to deliver the desired benefit. An effective amount is an adequate amount to deliver the desired benefit but low enough to avoid serious side effects at a reasonable benefit to risk ratio within the scope of sound medical judgment. What is a safe and effective amount of the active ingredients will vary with the specific active, the ability of the active to penetrate through the skin, the age of the user, the health condition of the user, the skin condition of the user and other like factors. Such methods comprise topically applying to the skin an effective amount of the composition of the present invention.

The compositions of the invention may be applied directly to the skin or by the use of transdermal treatment systems, including patches, which are semi permeable membranes with the active compound applied to a top surface thereof.

A common type of transdermal treatment systems, also variously referred to as a medical bandage, treatment pad, drug patch, etc., includes a drug reservoir or depot in the form of a drug-storing matrix or carrier and means for attaching or securing the device to a surface of unbroken skin. Representative transdermal treatment systems are described in, among others, U.S. Pat. Nos. 3,797,494; 3,996,934; 4,060,084; 4,286,592; 4,379,454; 4,568,343; 4,588,400; 4,573,995; 4,615,599; 4,764,379; 4,863,738; 5,006,342; and, 5,066,494.

It will be found in practice that the compositions of the invention may be used to target any tissue area included in the epidermis, dermis, subcutaneous tissue, fascia, or muscle.

The invention further relates to the a method of administering topical anesthetic agents along with a vasodilator comprising the steps of:

a. providing a composition comprising a therapeutically safe and effective amount of at least one topical anesthetic; a therapeutically safe and effective amount of at least one vasodilator; and a pharmaceutically acceptable carrier; and

b. contacting an area of skin of a subject with the composition thereby administering the topical anesthetic and vasodilator, preferably at the site of insertion of an intravenous device.

The term “administering” is intended to mean any mode of application that results in the physical contact of the composition with an anatomical site in need of treatment. The term “subject” is intended to include all warm-blooded mammals, preferably humans.

In another embodiment, the present invention further comprises:

c. waiting an appropriate time for the composition to provide its anesthetic and vasodilation effect; and

d. inserting a medical device through the skin or dermis at the site of application.

Generally, the composition is allowed to act from about 5 to about 90 minutes before inserting a device at the site of application. Preferably, the composition is allowed to act from about 15 to about 90 minutes and more preferably composition is allowed to act from about 30 to about 60 minutes.

In yet another embodiment, the present invention also encompasses a method of treatment to the skin of a mammal comprising the steps of applying a therapeutically safe and effective amount of the anesthetic and vasodilator composition at the site of interest for inserting an intravenous or intravascular device such as a needle, catheter, cannula, tube, syringe, stint or other insertable medical device that pierces the skin or dermis.

Generally, the composition will be applied and allowed to act for an appropriate time for the onset of anesthesia and vasodilation. The term “onset of anesthesia” is intended to mean to time to peak effect on the individual nerves. The onset of anesthesia principally depends upon the lipid solubility molecular size and quantity of active agent available. The duration of effect is the period of time during which the anesthetic measurably blocks nerve conduction.

The following examples will further describe the instant invention and are used for the purposes of illustrations only and should not be considered as limiting in any way the invention being disclosed herein. Percent (%) as used in these examples refer to percentage of the liquid formulation on a weight to weight basis and temperatures are given in degree C.

### A. EXAMPLE 1

| Ingredients | Percent (w/w) |
|---|---|
| Anesthetic agent base (lidocaine base) | 2.5 |
| Anesthetic agent sale (prilocaine | 2.5 |
| hydrochloride) | |
| Vasodilator (1,2,3-propanetriol trinitrate) | 0.25 |
| Carrier and other ingredients | 94.75 |

### B. EXAMPLE 2

| Ingredients | Percent (w/w) |
|---|---|
| Anesthetic agent base (lidocaine base) | 2.5 |
| Anesthetic agent sale (prilocaine | 2.5 |
| hydrochloride) | |
| Vasodilator (1,2,3-propanetriol trinitrate) | 0.125 |
| Carrier | 94.875 |

### C. EXAMPLE 3

| Ingredients | Percent (w/w) |
|---|---|
| Anesthetic agent base (lidocaine base) | 4.5 |
| Anesthetic agent sale (prilocaine | 0.5 |
| hydrochloride) | |
| Vasodilator (1,2,3-propanetriol trinitrate) | 0.2 |
| Carrier | 94.8 |

### D. EXAMPLE 4

| Ingredients | Percent (w/w) |
|---|---|
| Anesthetic agent base (lidocaine base) | 3.5 |
| Anesthetic agent sale (prilocaine | 2.0 |
| hydrochloride) | |
| Anesthetic agent sale (dibucaine) | 1.5 |
| Vasodilator (1,2,3-propanetriol trinitrate) | 0.25 |
| Carrier | 92.75 |

### E. EXAMPLE 5

| Ingredients | Percent (w/w) |
|---|---|
| Anesthetic agent base (lidocaine base) | 2.5 |
| Anesthetic agent sale (prilocaine | 2.5 |
| hydrochloride) | |
| Vasodilator (1,2,3-propanetriol trinitrate) | 0.2 |
| Polyoxyethylene fatty acid esters | 2 |
| Carboxypolymethylene | 0.7 |
| Sodium hydroxide | 0.1 |
| Water | 92 |

### F. EXAMPLE 6

| Ingredients | Percent (w/w) |
|---|---|
| Anesthetic agent base (lidocaine base) | 12 |
| Anesthetic agent sale (tetracaine | 12 |
| hydrochloride) | |
| Vasodilator (papaverine) | 0.2 |
| PEG-80 | 3 |
| Hydroxyethylcellulose | 0.5 |
| PPG-20 | 0.5 |
| Lipophilic base | 71.8 |

The foregoing examples are illustrative embodiments of the invention and are merely exemplary. A person skilled in the art may make variations and modification without departing from the spirit and scope of the invention. All such modifications and variations are intended to be included within the scope of the invention as described in the specification and the appended claims.

A preferred composition is made by mixing about 5 g. EMLA Cream (250 mg of a 1:1 by weight mixture of lidocaine and prilocaine emulsified in an aqueous vehicle) together with about 6 mg. of nitroglycerine. In use, about 125 mg. of the anesthetic active mixture and about 3 mg of nitroglycerine are applied to a skin area of about 5 cm², under occlusion, for a period of from about 30 to about 60 minutes. The dosage of the vasodilator component is somewhat lower than what would typically be used because the goal is to achieve the vasodilation effect in the skin and not to achieve any systemic effects. In fact, systemic effects should be minimized. Generally, the vasodilator, in the form of nitroglycerine, is applied at a level of up to about 80 μg/kg. The lidocaine/prilocaine mixture is generally applied at a level of from about 0.5 g to about 5 g.

When the compositions of the present invention are utilized, as described above, the patients show better color and improved venous dilation at the site of application, when compared to patients who are treated with the anesthetic alone. Additionally, the patients who utilize the present invention show improved access for placement of IV lines, and the insertion can be accomplished without pain. The active ingredients do not show any negative interactions upon mixing. Further, the inclusion of the vasodilator presents the opportunity to get the full anesthetic effect for the patient in less time (i.e., less than one hour) than is typically required for topical anesthetics.

What is claimed is:

1. A local, topical transdermal anesthetic and vasodilator formulation comprising a therapeutically safe and effective amount of:
a. at least one pharmaceutically active topical anesthetic;
b. at least one pharmaceutically active topical vasodilator; and
c. a pharmaceutically acceptable carrier.

2. The formulation of claim 1, wherein the topical anesthetic is selected from the group consisting of benzocaine, bupivacaine, butamben, butambenpicrate, chloroprocaine hydrochloride, chlorprocaine, cocaine, cocaine hydrochloride, dibucaine, dibucaine hydrochloride, dimethisoquin hydrochloride, diperodon, diphenhydramine, dyclonine, dyclonine hydrochloride, etidocaine, hexylcaine, ketamine, lidocaine, lidocaine hydrochloride, mepivacaine, methapyriline, oxyprocaine hydrochloride, p-buthylaminobenzoic acid 2-(die-ethylamino) ethyl ester hydrochloride, phenol, piperocaine hydrochloride, pramoxine, pramoxine hydrochloride., procaine, procaine hydrochloride, tetracaine, tetracaine hydrochloride, tripelennamine, and pharmaceutically acceptable acids, bases, and salts thereof.

3. The formulation of claim 1, wherein the topical anesthetic is at least one pharmaceutically active anesthetic selected from the group consisting of benzocaine, lidocaine, bupivacaine, dibucaine, mepivacaine, etidocaine, butanilicaine, prilocaine, tetracaine and trimecaine and their salts thereof.

4. The formulation of claim 1, wherein the topical anesthetic is at least one pharmaceutically active anesthetic selected from the group consisting of lidocaine, dibucaine, tetracaine, and prilocaine and their salts thereof.

5. The formulation of claim 2, wherein the topical anesthetic agent comprises a eutectic mixture.

6. The formulation of claim 5, wherein the topical anesthetic agent comprises a eutectic mixture of lidocaine and prilocaine.

7. The formulation of claim 1, wherein the anesthetic agent comprises a eutectic mixture consisting essentially of prilocaine in the form of its base in admixture with lidocaine, in the form of its base in a weight ratio of from about 1:4 to about 4:1.

8. The formulation of claim 1, wherein the topical vasodilator is at least one pharmaceutically active anesthetic selected from the group consisting of organic nitrates, glyceryl trinitrate, adenine, arginine, phentolamine, nicotinates, prostaglandins (eicosanoids), calcium antagonists, papaverine, nimodipine, hydralazine, nitric oxide, epoprostenol, tolazoline, aminone, milrinone, nitroglycerine, isosorbide dinitrate, isosorbide mononitrate, erythritol tetranitrate, pentaerythritol tetranitrate, dipyridamole, dilazep, trapidil, trimetazidine, and their substituted derivatives.

9. The formulation of claim 8, wherein the organic nitrate is selected from the group consisting of nitroglycerine, tetranitroerythritol, hexanitroinositol, tetranitropentaerythritol, propatyl nitrate, isosorbide 5-mononitrate (IS-5-MN), isosorbide dinitrate, isosorbide 2-mononitrate (IS-2-MN), isomannide 2-nitrate, 1,4:3,6-dianhydrohexitol nitrates, and trinitrotriethanolamine, and their substituted derivatives.

10. The formulation of claim 9, wherein the organic nitrate is nitroglycerine.

11. The formulation of claim 8, wherein the topical anesthetic comprises from about 0.5 to about 30% and the vasodilator comprises from about 0.01 to about 5% by weight based upon the total weight of the formulation.

12. The formulation of claim 8, wherein the topical anesthetic comprises from about 0.5 to about 20% and the vasodilator comprises from about 0.01 to about 2% by weight based upon the total weight of the formulation.

13. The formulation of claim 8, wherein the topical anesthetic comprises from about 0.5 to about 10% and the vasodilator comprises from about 0.01 to about 1% by weight based upon the total weight of the formulation.

14. The formulation of claim 1 or 8, wherein the pharmaceutically acceptable carrier is selected from the group consisting of a gel, paste, foam, ointment, cream, lotion, liquid suspension, solution, spray, emulsion, liposomes, film, laminate, and transdermal treatment system.

15. The formulation of claim 1 or 14, wherein the formulation further comprises at least one dermatologically acceptable ingredient selected from the group consisting of emollients, emulsifiers, thickening agents, buffers, solvents, mixed solvents, preservatives, coloring agents, fragrances, anti-oxidants, preservatives antimicrobial and antifangal actives, anti-inflammatory actives, lower alcohols, polyols, esters of fatty acids, oils, and waxes, silicones, antifoam agents, hydrating agents, stabilizers, surfactants, fillers, sequestrants, anionic, cationic, nonionic and amphoteric polymers, propellants, alkalifying and acidifying agents and mixtures thereof.

16. The formulation of claim 14, wherein the formulation further comprises a emollient.

17. The formulation of claim 14, wherein the formulation further comprises from about 1% to about 20% of emollient and from about 50% to about 90% water.

18. The formulation of claim 14, wherein the formulation further comprises from about 5% to about 10%, of emollient and from about 60% to about 80%, water.

19. The formulation of claim 14, wherein the formulation further comprises from about 5% to about 50% of emollient and from about 45% to about 85% water.

20. The formulation of claim 14, wherein the formulation further comprises from about 10% to about 20% of emollient and from about 50% to about 75%, water.

21. The formulation of claim 14, wherein the formulation further comprises a pharmaceutically acceptable thickening agent.

22. The formulation of claim 14, wherein the formulation is an ointment further comprising from about 2% to about 10% of an emollient, and from about 0.1% to about 2% of a thickening agent.

23. The formulation of claim 14, wherein the formulation further comprises a pharmaceutically acceptable thickening agent.

24. The formulation of claim 23, wherein the thickening agent is selected from the group consisting of CARBOPOL, HYPAN, NATROSOL, KLUCEL, STABILEZE, carboxypolymethylene, hydroxyethylcellulose, cellulose gum, MVE/MA decadiene crosspolymer, PVM/MA copolymer, block copolymers of polyoxyethylene and polyoxypropylene, or combinations thereof.

25. The formulation of claim 24, wherein the thickening agent concentration of the formulation is from about 0.1% to about 10%.

26. The formulation of claim 24, wherein the thickening agent concentration of the formulation is from about 0.1% to about 5%.

27. The formulation of claim 24, wherein the thickening agent concentration of the formulation is from about 0.2% to about 3%.

28. The formulation of claim 14, wherein the formulation further comprises a pharmaceutically acceptable emulsifier.

29. The formulation of claim 28, wherein the emulsifier is selected from the group consisting of alkylene oxide esters of fatty acids, alkylene oxide diesters of fatty acids, alkylene oxide ethers of fatty alcohols, the condensation products of alkylene oxides with both fatty acids and fatty alcohols, sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, and mixtures thereof.

30. The formulation of claim 28, wherein the emulsifier is selected from the group consisting of decyl polyglucoside and lauryl polyglucoside, ceteth-6, ceteth-10, ceteth-12, ceteareth-6, ceteareth-10, ceteareth-12, steareth-6, steareth-10, steareth-12, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof.

31. The formulation of claim 28, wherein the emulsifier concentration of the formulation is from about 0.05% to about 10%.

32. The formulation of claim 28, wherein the emulsifier concentration of the formulation is from about 0.1% to about 6%.

33. The formulation of claim 28, wherein the emulsifier concentration of the formulation is from about 0.1% to about 3% of at least one surfactant.

34. A local, topical transdernal anesthetic and vasodilator formulation, comprising a eutectic mixture of at least one first pharmaceutically active anesthetic, at least one second pharmaceutically active anesthetic in a ratio of from about 15:1 to about 15:1, and at least one pharmaceutically active vasodilator, in a suitable pharmaceutically acceptable carrier, wherein
(a) said at least one first pharmaceutically active anesthetic is selected from the group consisting of benzocaine, lidocaine, bupivacaine, dibucaine, mepivacaine, etidocaine, tetracaine, butanilicaine and trimecaine
(b) said at least one second pharmaceutically active anesthetic is selected from the group consisting of prilocaine, tetracaine, butanilicaine and trimecaine, and
(c) said at least one first pharmaceutically active anesthetic is different than said at least one second pharmaceutically active anesthetic.

35. The formulation of claim 34, wherein the topical vasodilator is at least one pharmaceutically active anesthetic selected from the group consisting of organic nitrates, glyceryl trinitrate, adenine, arginine, phentolamine, nicotinates, prostaglandins (eicosanoids), calcium antagonists, papaverine, nimodipine, hydralazine, nitric oxide, epoprostenol, tolazoline, arninone, milrinone, nitroglycerine, isosorbide dinitrate, isosorbide mononitrate, erythritol tetranitrate, pentaerythritol tetranitrate, dipyridamole, dilazep, trapidil, trimetazidine, and their substituted derivatives.

36. The formulation of claim 35, wherein the organic nitrate is selected from the group consisting of nitroglycerine, tetranitroerythritol, hexanitroinositol, tetranitropentaerythritol, propatyl nitrate, isosorbide 5-mononitrate (IS-5-MN), isosorbide dinitrate, isosorbide 2-mononitrate (IS-2-MN), isomannide 2-nitrate, 1,4:3,6-dianhydrohexitol nitrates, and trinitrotriethanolamine, and their substituted derivatives.

37. The formulation of claim 36, wherein the organic nitrate is nitroglycerine.

38. The formulation of claim 34, wherein the eutectic mixture of at least one first pharmaceutically active anesthetic and at least one second pharmaceutically active anesthetic are present in a ratio of from about 10:1 to about 1:10.

39. The formulation of claim 34, wherein the eutectic mixture of at least one first pharmaceutically active anesthetic and at least one second pharmaceutically active anesthetic are present in a ratio of from about 4:1 to about 1:4.

40. The formulation of claim 34, wherein the eutectic mixture comprises lidocaine and prilocaine in a lipophilic base.

41. The formulation of claim 40, wherein said lipophilic base is a petroleum product comprises from about 30% to about 90% by weight of the formulation.

42. The formulation of claim 41, wherein said lipophilic base is a higher aliphatic alcohol of 8-18 carbon atoms, or an ester thereof.

43. The formulation of claim 41, wherein said lipophilic base is a paraffin.

44. The formulation of claim 35, wherein the eutectic mixture comprises lidocaine and prilocaine in a weight ratio of from about 15:1 to about 1:15.

45. The formulation of claim 35, wherein the eutectic mixture comprises lidocaine and prilocaine in a weight ratio of from about 4:1 to about 1:4.

46. The formulation of claim 35, wherein the topical anesthetic comprises from about 0.5 to about 30% and the vasodilator comprises from about 0.01 to about 5% by weight based upon the total weight of the formulation.

47. The formulation of claim 35, wherein the topical anesthetic comprises from about 0.5 to about 20% and the vasodilator comprises from about 0.01 to about 2% by weight based upon the total weight of the formulation.

48. The formulation of claim 35, wherein the topical anesthetic comprises from about 0.5 to about 10% and the vasodilator comprises from about 0.01 to about 1% by weight based upon the total weight of the formulation.

49. The formulation of claim 35, further comprising dibucaine from 0.2 to 2% by weight of the total formulation.

50. The formulation of claim 35, wherein the pharmaceutically acceptable carrier is selected from the group consisting of a gel, paste, foam, ointment, cream, lotion, liquid suspension, solution, spray, emulsion, liposomes, film and laminate.

51. The formulation of claim 50, wherein the formulation further comprises at least one dermatologically acceptable ingredient selected from the group consisting of emollients, emulsifiers, thickening agents, buffers, solvents, mixed solvents, preservatives, coloring agents, fragrances, anti-oxidants, preservatives antimicrobial and antifungal actives, anti-inflammatory actives, lower alcohols, polyols, esters of fatty acids, oils, and waxes, silicones, antifoam agents, hydrating agents, stabilizers, surfactants, fillers, sequestrants, anionic, cationic, nonionic and amphoteric polymers, propellants, alkalifying and acidifying agents and mixtures thereof.

52. The formulation of claim 51, wherein the formulation further comprises an emollient.

53. The formulation of claim 52, wherein the formulation further comprises from about 1% to about 20% of emollient and from about 50% to about 90% water.

54. The formulation of claim 52, wherein the formulation further comprises from about 5% to about 10%, of emollient and from about 60% to about 80%, water.

55. The formulation of claim 52, wherein the formulation further comprises from about 5% to about 50% of emollient and from about 45% to about 85% water.

56. The formulation of claim 52, wherein the formulation further comprises from about 10% to about 20% of emollient and from about 50% to about 75%, water.

57. The formulation of claim 52, wherein the formulation further comprises a pharmaceutically acceptable thickening agent.

58. The formulation of claim 57, wherein the formulation is an ointment further comprising from about 2% to about 10% of an emollient, and from about 0.1% to about 2% of a thickening agent.

59. The formulation of claim 57, wherein the formulation further comprises a pharmaceutically acceptable thickening agent.

60. The formulation of claim 57, wherein the thickening agent is selected from the group consisting of CARBOPOL, HYPAN, NATROSOL, KLUCEL, STABILEZE, carboxypolymethylene, hydroxyethylcellulose, cellulose gum, MVE/MA decadiene crosspolymer, PVM/MA copolymer, block copolymers of polyoxyethylene and polyoxypropylene, or combinations thereof.

61. The formulation of claim 57, wherein the thickening agent concentration of the formulation is from about 0.1% to about 10%.

62. The formulation of claim 57, wherein the thickening agent concentration of the formulation is from about 0.1% to about 5%.

63. The formulation of claim 57, wherein the thickening agent concentration of the formulation is from about 0.2% to about 3%.

64. The formulation of claim 50, wherein the formulation further comprises a pharmaceutically acceptable emulsifier.

65. The formulation of claim 64, wherein the emulsifier is selected from the group consisting of alkylene oxide esters of fatty acids, alkylene oxide diesters of fatty acids, alkylene oxide ethers of fatty alcohols, the condensation products of alkylene oxides with both fatty acids and fatty alcohols, sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, and mixtures thereof.

66. The formulation of claim 64, wherein the emulsifier is selected from the group consisting of decyl polygluco side and lauryl polyglucoside, ceteth-6, ceteth-10, ceteth-12, ceteareth-6, ceteareth-10, ceteareth-12, steareth-6, steareth-10, steareth-12, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof.

67. The formulation of claim 64, wherein the emulsifier concentration of the formulation is from about 0.05% to about 10%.

68. The formulation of claim 64, wherein the emulsifier concentration of the formulation is from about 0.1% to about 6%.

69. The formulation of claim 64, wherein the emulsifier concentration of the formulation is from about 0.01% to about 3%.

70. The formulation of claim 52, wherein the topical anesthetic comprises a third anesthetic selected from the group consisting of benzocaine, bupivacaine, butamben, butambenpicrate, chloroprocaine hydrochloride, chlorprocaine, cocaine, cocaine hydrochloride, dibucaine, dibucaine hydrochloride, dimethisoquin hydrochloride, diperodon, diphenhydramine, dyclonine, dyclonine hydrochloride, etidocaine, hexylcaine, ketamine, lidocaine, lidocaine hydrochloride, mepivacaine, methapyriline, oxyprocaine hydrochloride, p-buthylaminobenzoic acid 2-(die-ethylamino) ethyl ester hydrochloride, phenol, piperocaine hydrochloride, pramoxine, pramoxine hydrochloride., procaine, procaine hydrochloride, tetracaine, tetracaine hydrochloride, tripelennamine, and pharmaceutically acceptable acids, bases, and salts thereof.

71. A method of administering topical anesthetic agents along with a vasodilator comprising the steps of:
(a) providing a composition comprising a therapeutically safe and effective amount of at least one topical anesthetic; a therapeutically safe and effective amount of at least one vasodilator; and a pharmaceutically acceptable carrier; and
(b) contacting an area of skin of a subject with the composition thereby administering the topical anesthetic and vasodilator, preferably at the site of insertion of an intravenous device.

72. The method of claim 71, wherein the method further comprises waiting an appropriate time for the composition to provide its anesthetic and vasodilation effect.

73. The method of claim 72, wherein the method further comprises inserting a medical device through the skin or dermis at the site of application.

74. The method of claim 71, wherein the composition is allowed to act from about 5 to about 90 minutes.

75. The method of claim 71, wherein the composition is allowed to act from about 15 to about 90 minutes.

76. The method of claim 71, wherein the composition is allowed to act from about 30 to about 60 minutes.

77. The method of claim 73, wherein the medical device is an intravenous or intravascular device selected from the group consisting of a needle, catheter, cannula, tube, syringe, stint or other insertable medical device that pierces the skin or dermis.
